(19) United States
(12) Patent Application Publication
(10) Pub. No.: US 2012/0085352 A1
(43) Pub. Date: Apr. 12, 2012

(54) MEDICAL SECURING DEVICE

(75) Inventor: Ian David Worthington, West Yorkshire (GB)

(73) Assignee: Kapitex Healthcare Limited, Wetherby (GB)

(21) Appl. No.: 13/378,437

(22) PCT Filed: Jun. 18, 2009
(86) PCT No.: PCT/GB09/01517

§ 371 Date: Dec. 15, 2011

(51) Int. Cl. A61M 16/04

(52) U.S. Cl. 128/207.17

(57) ABSTRACT

A medical securing device (1) comprising a flexible strap (2) including a longitudinal slot (4) proximal to a transversely narrow bridge section (3) disposed between distal end portions (6, 7), wherein the end portions are connectable to a medical neckband.

This invention relates to a medical securing device. More specifically, this invention relates to a device for securing a endotracheal tube to a neckband placed around the neck and head of a endotracheal patient.

Conventionally, a neckband is placed around the neck of a patient undergoing an endotracheal procedure. The endotracheal tube is inserted through a plastic clamping device to which the neckband is secured. Usually, the neckband is secured to the tube via hook and eye fixing strips or it is simply tied to the tube.

Endotracheal tubes are produced in a variety of sizes and are made from silicone, PVC, or TPE.

With the conventional method of securing an endotracheal tube to a patient, each size of endotracheal tube requires a separate neckband having a plastic clamping device of the correct size.

The present invention arose from an attempt to provide a universal endotracheal tube holder adapted to fit all available sizes of endotracheal tubes.

In accordance with an aspect of the present invention there is provided a medical securing device comprising a flexible strap including a longitudinal slot proximal to a transversely narrow bridge section disposed between distal end portions, wherein the end portions are connectable to a medical neckband.

The device is adapted to be tied around a endotracheal tube, and end portions of the flexible strap include a slot or eyelet for receiving loop or tie portions of the medical neckband.

Preferably, a central portion of the device includes the longitudinal slot and narrow bridge section. This central portion is disposed between twistable and flexible narrow sections that are interconnected with the end portions. Also, the narrow bridge section is wider than the twistable and flexible narrow sections. This enables the end portion to be threaded through the longitudinal slot so that the narrow bridge section forms a loop. The loop encompasses the endotracheal, thus enabling it to be secured to a patient's neckband.

Preferably, the device is fabricated from silicone, but it can alternatively be made from natural rubber, PVC, or TPE.

An embodiment of the present invention will now be described, by way of example only, and with reference to the accompanying schematic drawings, in which:

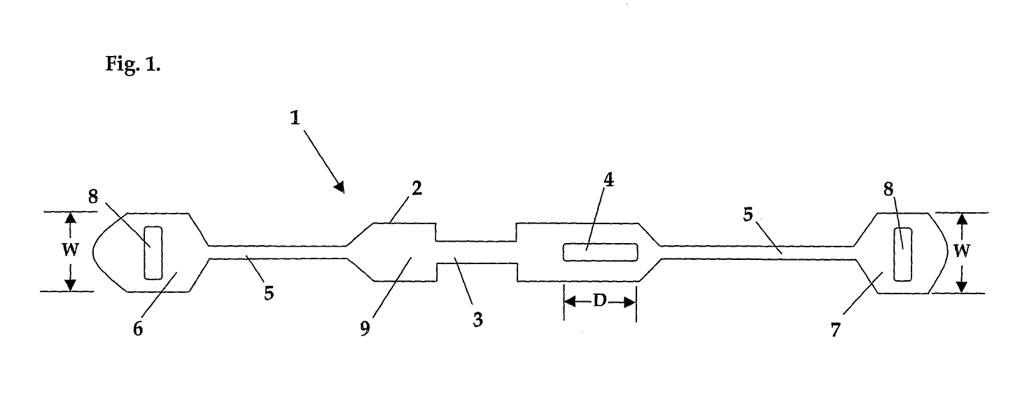

FIG. 1 shows an embodiment of a medical securing device in plan view; and

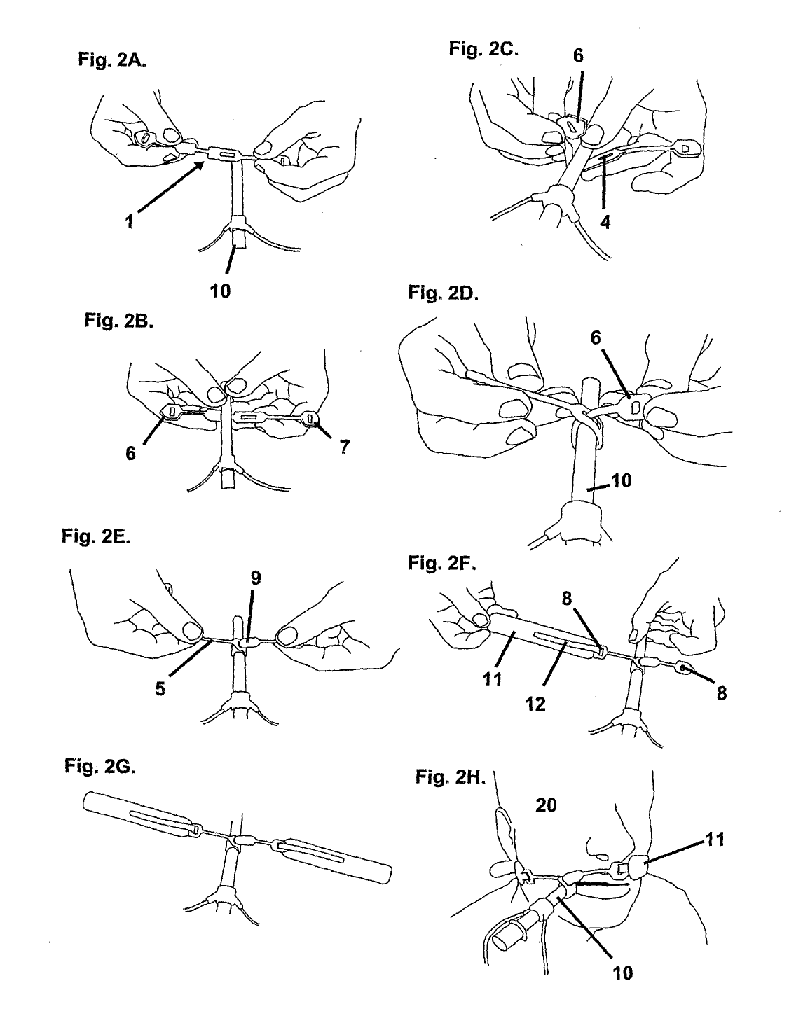

FIGS. 2A to 2H depict the steps of securing the medical securing device of FIG. 1 to a endotracheal tube.

Referring to FIG. 1, a medical securing device 1 comprises a profiled silicone strap 2 having end portions 6, 7. Each end portion 6, 7 includes a slot 8 for receiving a loop portion or tie strip of a patient neckband [see FIG. 2G].

A central portion of the medical securing device 1 includes a bridge section 3, a longitudinal slot 4, and a locking portion 9. The bridge section 3 is transversely narrow in comparison with the width W of either of the end portions 6, 7. The width

W is greater than the length D of the longitudinal slot 4. However, since the device is constructed from silicone, and is therefore flexible, end portion 6 (which has a slightly pointed profile) can be resiliently forced through the longitudinal slot 4. It should be noted that this same operation is possible with a rubber or PVC securing device.

The end portions 6, 7 are connected to the aforementioned central portion by narrow flexible interconnect sections 5. The interconnect sections 5 are twistable with respect to both the end portions 6, 7 and to the central portion.

A method of securing a endotracheal tube to a patient neckband using the medical securing device of the present invention is shown in FIGS. 2A to 2H.

As shown in FIGS. 2A and 2B, a medical securing device 1 is brought into contact with a endotracheal tube 10. Then, as shown in FIGS. 2C and 2D, end portion 6 is looped around the endotracheal tube 10, and threaded through the longitudinal slot 4 by twisting the corresponding interconnect section 5 so that the end portion 6 and longitudinal slot 4 are in alignment.

As shown in FIG. 2E, end portion 6 is pulled through the slot 4 and away from the endotracheal tube until the locking portion 9 is also pulled through the longitudinal slot 4. After the locking portion 9 passes through the longitudinal slot 4, rearward shoulder sections of the locking portion 9 prevent rearward movement of the locking portion 9, thus locking the medical securing device 1 around the endotracheal tube 10.

Once the medical securing device 1 is secured in place around the endotracheal tube 10 as shown in FIG. 2E, tie portions 12 of a two-piece patient neckband 11 are looped through end portion slots 8 and secured to the neckband 11 using conventional hook and eye material.

As shown in FIG. 2H, once correctly inserted into a patient, the endotracheal tube 10 is held securely in place by virtue of the two-piece neckband 11 and the medical securing device 1.

1. A medical securing device comprising a flexible strap including a longitudinal slot proximal to a transversely narrow bridge section disposed between distal end portions, wherein the end portions are connectable to a medical neckband.

2. A medical securing device as claimed in claim 1, wherein the device is adapted to be tied around a endotracheal tube.

3. A medical securing device as claimed in claim 1, wherein the end portions include a slot or eyelet for receiving loop portions of the medical neckband.

4. A medical securing device as claimed in claim 1, wherein a central portion of the device includes said longitudinal slot and said narrow bridge section, and said central portion is disposed between twistable and flexible narrow sections that are interconnected with the end portions.

5. A medical securing device as claimed in claim 4, wherein said narrow bridge section is wider than said twistable and flexible narrow sections.

6. A medical securing device as claimed in claim 1, wherein the device is fabricated from silicone.

7. A medical securing device as claimed in claim 1, wherein the device is fabricated from a member selected from the group consisting of natural rubber, PVC, and TPE.

8. A medical securing device as claimed in claim 1, wherein at least one end portion is threadable through the longitudinal slot to form a loop.

9. A medical securing device as claimed in claim 8, wherein the narrow bridge section is adapted to be looped around the endotracheal tube.
